(19) United States
(12) Patent Application Publication
(10) Pub. No.: US 2023/0220428 A1
(43) Pub. Date: Jul. 13, 2023

(54) YEAST STRAIN AND USE THEREOF AND PREPARATION METHOD OF ERGOTHIONEINE

(71) Applicant: XIAMEN OAMIC BIOTECH CO., LTD., Xiamen (CN)

(72) Inventors: Liqin Zhou, Xiamen (CN); Ting Xiang, Xiamen (CN); Meixue Yang, Xiamen (CN); Wenxu Peng, Xiamen (CN); Chenguang Xing, Xiamen (CN); Gang Liu, Xiamen (CN)

(21) Appl. No.: 17/733,795

(22) Filed: Apr. 29, 2022

(30) Foreign Application Priority Data
Jan. 7, 2022 (CN) 202210017520.8

(51) Int. Cl. C12P 13/04; C12P 17/10; C12N 1/02; C12N 1/18

(52) U.S. Cl. CPC C12P 13/04; C12P 17/10; C12N 1/02; C12N 1/185; C12N 1/005

(57) ABSTRACT

The present relates to a yeast strain and use thereof and a preparation method of ergothioneine. The present invention relates to the field of biotechnology. The yeast strain is obtained through traditional mutagenesis and screening, and its deposit number is CCTCC M 20211505. The present invention provides a preparation method of ergothioneine. The preparation method of ergothioneine comprises: mixing the aforementioned yeast strain with a fermentation medium and an optional substrate, fermenting, and then homogenizing cells and separating to obtain ergothioneine. The aforementioned yeast strain can be used for the preparation of ergothioneine, and the ergothioneine prepared by the yeast strain has the advantages of high yield, low cost and fast preparation speed. The preparation method has the advantages of low cost, environmental protection, high product quality, high yield, less impurities, less drug residues, short fermentation period and the like.

### CROSS REFERENCE TO RELATED APPLICATION

The present application claims priority from Chinese Patent Application No. 202210017520.8 filed on Jan. 7, 2022, the contents of which are incorporated herein by reference in their entirety.

### TECHNICAL FIELD

The present invention relates to the field of biotechnology. Specifically, it relates to a yeast strain and use thereof and a preparation method of ergothioneine.

### BACKGROUND OF THE INVENTION

Ergothioneine is a naturally occurring amino acid and is a thiourea derivative of histidine, containing a sulfur atom on the imidazole ring. Ergothioneine was isolated from Claviceps purpurea by MC Tanret in 1909 and named after the ergot fungus from which it was first purified, with its structure being determined in 1911. Ergothioneine is a water-soluble substance that is easily soluble in water and has excellent thermal stability and pH stability. Ergothioneine is also a natural substance with strong antioxidant activity, and has the characteristics of being hardly affected by acidic (pH 2) to alkaline (pH 12) conditions. In humans, ergothioneine is obtained entirely through diet and accumulates in red blood cells, bone marrow, livers, kidneys, semen, and eyes.

Basidiomycetes such as mushrooms and some bacteria can biosynthesize ergothioneine. Mushrooms are ergothioneine-rich foods. Ergothioneine is particularly abundant in Pleurotus ostreatus. Actinomycetes (such as Mycobacterium smegmatis) and certain fungi (such as Neurospora crassa) can also biosynthesize ergothioneine. The metabolic pathway of ergothioneine starts with the methylation of histidine to produce histidine betaine (Hercynine), and then a sulfur atom is introduced from cysteine to give ergothioneine. Other species of bacteria, such as Bacillus subtilis, Escherichia coli, Proteus vulgaris and Streptococcus, and fungi such as budding yeast strains cannot biosynthesize ergothioneine.

At present, there are three methods for preparing ergothioneine: chemical synthesis, extraction and bio-fermentation synthesis. In the process of realizing the present invention, the inventors found that there are at least the following problems in the prior art: it is very difficult to synthesize L-ergothioneine by chemical methods. The difficulty lies in the preparation of the raw material 2-mercaptoimidazole, and the acidity of the a-carbon makes the reaction prone to partial or full racemization. Due to the expensive raw materials and high synthesis cost, the price of the product is high, which limits the application of ergothioneine. The natural biological extraction method is to extract ergothioneine from the fruiting bodies of edible fungi, pig blood, animal tissue, ergot and grains, but the content of ergothioneine in the above-mentioned raw materials is very low, and there are problems such as many impurities in the raw materials, drug residues, and high extraction cost, which are not conducive to the industrialization of ergothioneine. The biological preparation of ergothioneine by submerged fermentation technology of mushroom mycelium is the mainstream direction of large-scale production of ergothioneine with low cost. It can effectively increase the yield of ergothioneine and reduce production cost through fermentation process control methods such as metabolic regulation, and more importantly, the safety of the product can be guaranteed and the application space of ergothioneine can be broadened. However, the production of ergothioneine by submerged fermentation technology of mushroom mycelium has a long fermentation cycle and the maximum yield is only 135.7 mg/L (Jiang Wenxia, Liu Qi, Zhou Tao. Strains for producing ergothioneine and methods for preparing ergothioneine [P].CN201210392417.8). To date, the large-scale biosynthesis of natural ergothioneine has not been achieved in industry.

Therefore, there is still an urgent need for a preparation method of natural ergothioneine with low cost, high yield and short fermentation cycle.

### SUMMARY OF THE INVENTION

In order to solve the above-mentioned problems, the present invention provides a yeast strain and use thereof and a preparation method of ergothioneine.

In the first aspect, the present invention provides a yeast strain. The yeast strain is obtained through traditional mutagenesis and screening, and its deposit number is CCTCC M 20211505; the yeast strain has been deposited biologically at China Center for Type Culture Collection (CCTCC, Wuhan University) on Nov. 29, 2021, and is classified and named as Schizosaccharomyces pombe. The yeast strain can be used for preparing ergothioneine, and has the advantages of high yield, low cost, fast preparation speed and the like.

In the second aspect, the present invention provides a preparation method of ergothioneine. The preparation method of ergothioneine comprises: mixing the aforementioned yeast strain with a fermentation medium and an optional substrate, fermenting, and then homogenizing cells and separating to obtain ergothioneine. The aforementioned yeast strain can be used for the preparation of ergothioneine, and the ergothioneine prepared by the yeast strain has the advantages of high yield, low cost and fast preparation speed. The preparation method has the advantages of low cost, environmental protection, high product quality, high yield, less impurities, less drug residues, short fermentation period and the like.

In the third aspect, the present invention provides an ergothioneine prepared by the aforementioned preparation method. The ergothioneine has fewer impurities, less drug residues, less chemical residues, and high product quality.

In a fourth aspect, the present invention provides a method of preparing ergothioneine comprising using the yeast strain described in the first aspect.

### DETAILED DESCRIPTION OF THE INVENTION

In order to solve the above problems, the present invention provides the following technical solutions.

In the first aspect, the present invention provides a yeast strain.

A yeast strain has the deposit number of CCTCC M 20211505; the yeast strain has been deposited biologically at China Center for Type Culture Collection (CCTCC, Wuhan University) on Nov. 29, 2021, and is classified and named as Schizosaccharomyces pombe.

In one embodiment, a yeast strain capable of large-scale biosynthesis of natural ergothioneine is provided. In this application, yeast strains with high-yield natural ergothioneine are selected mainly through traditional mutagenesis with combined treatments of physical and chemical mutagenic agents and one of the preferred strains is named OMK-79. The yeast strain has been deposited biologically at China Center for Type Culture Collection (CCTCC, Wuhan University) on Nov. 29, 2021, and is classified and named as Schizosaccharomyces pombe, and the deposit number is CCTCC M 20211505.

In the second aspect, the present invention provides a preparation method of ergothioneine.

A preparation method of ergothioneine comprises: mixing the yeast strain described in the first aspect with a fermentation medium and an optional substrate, fermenting, and then homogenizing cells and separating to obtain ergothioneine. In some embodiments, a preparation method of ergothioneine comprises: inoculating the yeast strain described in the first aspect into a seed liquid culture medium for amplification culture to obtain a seed liquid, mixing the seed liquid with a fermentation medium and an optional substrate, fermenting, and then homogenizing cells and separating to obtain ergothioneine. In some embodiments, a preparation method of ergothioneine comprises: activating the yeast strain described in the first aspect to obtain a culture medium containing the activated yeast strain, inoculating the culture medium containing the activated yeast strain into a seed liquid culture medium for amplification culture to obtain a seed liquid, and mixing the seed liquid with a fermentation medium and an optional substrate, fermenting, and then homogenizing cells and separating to obtain ergothioneine.

The activation may includes inoculating the yeast strain described in the first aspect into an activation medium, and growing it at 20° C. to 40° C. (such as 20° C., 25° C., 30° C., 35° C. or 40° C.) and a rotation speed of 100 rpm-400 rpm (such as 100 rpm, 150 rpm, 200 rpm, 250 rpm, 300 rpm, 350 rpm or 400 rpm) for 16 hours to 60 hours (such as 16 hours, 17 hours, 18 hours, 19 hours, 20 hours, 21 hours, 22 hours, 23 hours, 24 hours, 30 hours, 35 hours, 40 hours, 45 hours, 48 hours, 50 hours, 55 hours or 60 hours). In some embodiments, the activation includes inoculating the yeast strain described in the first aspect into an activation medium and growing it at 25° C. to 35° C. and a rotation speed of 150 rpm-300 rpm for 24 hours to 48 hours.

The activation medium may comprise yeast extract, peptone, and glucose. In some embodiments, the activation medium comprises yeast extract 3 g/L to 30 g/L, peptone 3 g/L to 30 g/L, and glucose 5 g/L to 50 g/L. In some embodiments, the activation medium comprises yeast extract 3 g/L to 30 g/L, peptone 3 g/L to 30 g/L, glucose 5 g/L to 50 g/L, and the solvent is water. In some embodiments, the activation medium comprises yeast extract 10 g/L, peptone 10 g/L, and glucose 20 g/L. In some embodiments, the activation medium comprises yeast extract 10 g/L, peptone 10 g/L and glucose 20 g/L, and the solvent is water.

The temperature of the amplification culture may be 20° C. to 40° C. In some embodiments, the temperature of the amplification culture is 25° C. to 35° C. In some embodiments, the temperature of the amplification culture is 25° C. to 30° C. In some embodiments, the temperature of the amplification culture is 25° C., 26° C., 27° C., 28° C., 29° C., 30° C., 31° C., 32° C., 33° C., 34° C., or 35° C.

The rotation speed of the amplification culture may be 100 rpm to 900 rpm. In some embodiments, the rotation speed of the amplification culture is 150 rpm to 600 rpm. In some embodiments, the rotation speed of the amplification culture is 200 rpm to 500 rpm. In some embodiments, the rotation speed of the amplification culture is 100 rpm, 150 rpm, 200 rpm, 250 rpm, 300 rpm, 350 rpm, 400 rpm, 450 rpm, 500 rpm, 550 rpm, 600 rpm, 700 rpm, 800 rpm or 900 rpm.

The time for the amplification culture may be 16 hours to 200 hours. In some embodiments, the time for the amplification culture is 16 hours to 100 hours. In some embodiments, the time for the amplification culture is 16 hours to 50 hours. In some embodiments, the time for the amplification culture is 16 hours to 48 hours. In some embodiments, the time for the amplification culture is 16 hours, 17 hours, 18 hours, 19 hours, 20 hours, 21 hours, 22 hours, 23 hours, 24 hours, 30 hours, 35 hours, 40 hours, 45 hours or 48 hours.

The amplification culture comprises inoculating the culture medium containing the activated yeast strain into the seed liquid culture medium at an inoculation amount of 0.005% vol to 30% vol. Since the inoculation amount of the amplification culture can be adjusted in a wide range, and a very small inoculation amount can also be successfully amplified, which is common knowledge in the art. Therefore, the amplification culture may comprise inoculating the culture medium containing the activated yeast strain into the seed liquid culture medium at an inoculation amount of 0.005% vol to 30% vol. In some embodiments, the amplification culture comprises inoculating the culture medium containing the activated yeast strain into the seed liquid culture medium at an inoculation amount of 0.5% vol to 20% vol. In some embodiments, the amplification culture comprises inoculating the culture medium containing the activated yeast strain into the seed liquid culture medium at an inoculation amount of 1% vol to 10% vol. In some embodiments, the amplification culture comprises inoculating the culture medium containing the activated yeast strain into the seed liquid culture medium at an inoculation amount of 1% vol to 5% vol. In some embodiments, the amplification culture comprises inoculating the culture medium containing the activated yeast strain into the seed liquid culture medium at an inoculation amount of 1% vol, 2% vol, 3% vol, 4% vol, 5% vol, 6% vol, 7% vol, 8% vol, 9% vol, 10% vol, 15% vol, 20% vol, 25% vol or 30% vol.

The volume ratio of the seed liquid to the fermentation medium may be 1:200 to 3:10. In some embodiments, the volume ratio of the seed liquid to the fermentation medium is 1:150 to 2:10. In some embodiments, the volume ratio of the seed liquid to the fermentation medium is 1:100 to 1:10. In some embodiments, the volume ratio of the seed liquid to the fermentation medium is 1:100 to 1:20.

The added amount of the substrate can be 0-10 g/L; yeast strains can metabolize to produce substances such as arginine, histidine, methionine or cysteine, but additional substrates can increase the yield of ergothioneine. In some embodiments, the added amount of the substrate is 0, 0.5 g/L, 0.6 g/L, 0.7 g/L, 0.8 g/L, 0.9 g/L, 1.0 g/L, 1.2 g/L, 1.3 g/L, 1.4 g/L, 1.5 g/L, 2.0 g/L, 2.5 g/L, 3.0 g/L, 3.2 g/L, 3.5 g/L, 4.0 g/L, 4.5 g/L, 5.0 g/L, 5.5 g/L, 6.0 g/L, 6.5 g/L, 7.0 g/L, 7.5 g/L, 8.0 g/L, 8.5 g/L, 9.0 g/L, 9.5 g/L or 10.0 g/L.

The substrate may comprise at least one selected from arginine, histidine, methionine, or cysteine. In some embodiments, the substrate may comprise one selected from arginine, histidine, methionine, and cysteine.

The fermentation medium may comprise a carbon source.

The carbon source may comprise at least one selected from sucrose, fructose, xylose, ethanol, methanol, glycerol, glucose, cellulose, starch, cellobiose or other glucose-containing polymers. In some embodiments, the carbon source is glycerol.

In some embodiments, the preparation method comprises mixing the yeast strain described in the first aspect with a fermentation medium and an optional substrate, and growing under suitable culture conditions. Yeast strains can grow in batch fermentation, single fed-batch fermentation, mixed fed-batch fermentation, continuous fermentation, or a combination thereof.

In some embodiments, the substrate may be added to the fermentation system during the incubation period, exponential growth period, or plateau period of the yeast strain described in the first aspect.

The yeast strain described in the first aspect may be suspended or immobilized in the fermentation medium.

The fermentation medium may comprise phosphoric acid, CaSO₄, K₂SO₄, KOH, MgSO₄·7H₂O, glycerol, yeast extract, peptone, defoaming oil and sterile water. In some embodiments, the fermentation medium comprises 1% vol to 7% vol phosphoric acid, 0.03 wt % to 0.3 wt % CaSO₄, 1 wt % to 6 wt % K₂SO₄, 0.1 wt % to 2 wt % KOH, 0.5 wt % to 5 wt % MgSO₄·7H₂O, 1 wt % to 12 wt % glycerol, 0.1 wt % to 2 wt % yeast extract, 0.1 wt % to 2 wt % peptone, 0.01% vol to 1% vol defoaming oil, and the solvent is water. In some embodiments, the fermentation medium comprises 2.27% vol phosphoric acid, 0.093 wt % CaSO₄, 1.82 wt % K₂SO₄, 0.413 wt % KOH, 1.49 wt % MgSO₄·7H₂O, 4 wt % glycerol, 0.5 wt % yeast extract, 0.5 wt % peptone, 0.1% vol defoaming oil, and the solvent is water.

The seed liquid culture medium may comprise yeast extract, peptone, and glucose. In some embodiments, the seed liquid culture medium comprises yeast extract 3 g/L to 30 g/L, peptone 3 g/L to 30 g/L, and glucose 5 g/L to 50 g/L. In some embodiments, the seed liquid culture medium comprises yeast extract 3 g/L to 30 g/L, peptone 3 g/L to 30 g/L, and glucose 5 g/L to 50 g/L, and the solvent is water. In some embodiments, the seed liquid culture medium comprises yeast extract 10 g/L, peptone 10 g/L, and glucose 20 g/L. In some embodiments, the seed liquid culture medium comprises yeast extract 10 g/L, peptone 10 g/L, and glucose 20 g/L, and the solvent is water.

The fermentation temperature may be 20° C. to 40° C. In some embodiments, the fermentation temperature is 25° C. to 35° C. In some embodiments, the fermentation temperature is 25° C. to 30° C. In some embodiments, the fermentation temperature is 25° C., 26° C., 27° C., 28° C., 29° C., 30° C., 31° C., 32° C., 33° C., 34° C., or 35° C.

The fermentation time may be 16 hours to 200 hours. Generally speaking, a longer cultivation time produces a greater amount of natural ergothioneine. Therefore, the yeast strains can be cultured for 16 hours to 200 hours. In some embodiments, the fermentation time may be 24 hours to 200 hours. In some embodiments, the fermentation time may be 48 hours to 200 hours. In some embodiments, the fermentation time may be 100 hours to 200 hours. In some embodiments, the fermentation time may be 140 hours to 200 hours.

The airflow rate of the fermentation may be 0.8 L/min-8 L/min. In some embodiments, the airflow rate of the fermentation is 1 L/min to 7 L/min. In some embodiments, the airflow rate of the fermentation is 2 L/min to 6 L/min. In some embodiments, the airflow rate of the fermentation is 3 L/min to 5 L/min. In some embodiments, the airflow rate of the fermentation is 1 L/min, 2 L/min, 3 L/min, 4 L/min, 5 L/min, 6 L/min, 7 L/min, or 8 L/min.

The air pressure of the fermentation may be 0 to 0.6 MPa. In some embodiments, the air pressure of the fermentation is 0.05 MPa to 0.6 MPa. In some embodiments, the air pressure of the fermentation is 0.06 MPa to 0.50 MPa. In some embodiments, the air pressure of the fermentation is 0.07 MPa to 0.40 MPa. In some embodiments, the air pressure of the fermentation is 0.07 MPa to 0.30 MPa. In some embodiments, the air pressure of the fermentation is 0.07 MPa to 0.20 MPa. In some embodiments, the air pressure of the fermentation is 0.07 MPa to 0.10 MPa. In some embodiments, the air pressure of the fermentation is 0.05 MPa, 0.06 MPa, 0.07 MPa, 0.08 MPa, 0.09 MPa, 0.10 MPa, 0.15 MPa, 0.20 MPa, 0.25 MPa, 0.30 MPa, 0.35 MPa, 0.40 MPa, 0.50 MPa, or 0.6 MPa.

The pH of the fermentation system of the fermentation may be 4.0 to 7.0. In some embodiments, the pH of the fermentation system of the fermentation is 4.0, 4.5, 5.0, 5.5, 6.0, 6.5, or 7.0.

The pH of the fermentation system can be adjusted by adding ammonia water.

In some embodiments, the fermentation system refers to a system formed by mixing the aforementioned yeast strain with the substrates and the culture medium. In some embodiments, the fermentation system is a system formed by mixing the aforementioned yeast strain and the culture medium before the substrates are added. In some embodiments, the fermentation system is a system formed by mixing the aforementioned yeast strain and the culture medium.

The initial stirring speed of the fermentation may be between 100 rpm to 900 rpm. In some embodiments, the initial stirring speed of the fermentation is between 150 rpm to 600 rpm. In some embodiments, the initial stirring speed of the fermentation is between 200 rpm to 500 rpm. In some embodiments, the initial stirring speed of the fermentation is 100 rpm, 150 rpm, 200 rpm, 250 rpm, 300 rpm, 350 rpm, 400 rpm, 450 rpm, 500 rpm, 550 rpm, 600 rpm, 700 rpm, 800 rpm or 900 rpm.

In the fermentation step, when the dissolved oxygen concentration drops to 50%, glycerol is supplemented, the stirring speed is increased stepwise between 100 rpm to 600 rpm (for example, 100 rpm, 150 rpm, 200 rpm, 250 rpm, 300 rpm, 350 rpm, 400 rpm, 450 rpm, 500 rpm, 550 rpm or 600 rpm) based on the initial stirring speed, glycerol is supplemented continuously and the stirring speed maintains stepwise increment until the detected wet weight biomass reaches 30 g/L to 300 g/L (for example, 30 g/L, 50 g/L, 100 g/L, 150 g/L, 180 g/L, 190 g/L, 200 g/L, 210 g/L, 220 g/L, 250 g/L or 300 g/L). In some embodiments, in the fermentation step, the substrate can be added and glycerol is supplemented when the dissolved oxygen concentration drops to 50%, and the stirring speed is increased stepwise between 100 rpm to 600 rpm based on the initial stirring speed. Glycerol is supplemented continuously and the stirring speed maintains stepwise increment until the detected wet weight biomass reaches 30 g/L to 300 g/L.

The supplemented amount of glycerol may be 20 g/L to 220 g/L. In some embodiments, the supplemented amount of glycerol is 20 g/L, 30 g/L, 40 g/L, 50 g/L, 60 g/L, 70 g/L, 75 g/L, 80 g/L, 90 g/L, 100 g/L, 110 g/L, 120 g/L, 130 g/L, 140 g/L, 150 g/L, 160 g/L, 170 g/L, 180 g/L, 190 g/L, 200 g/L or 220 g/L.

The separation step may include separation and purification by centrifugation, extraction, vacuum distillation or resin adsorption, etc. In some embodiments, the separation step includes separation and purification by extraction, vacuum distillation or resin adsorption etc. to obtain homogeneous (for example, more than 90% HPLC purity) ergothioneine. In some embodiments, ergothioneine is isolated as an extract of yeast strains. In this case, ergothioneine can be isolated but need not be purified to homogeneity.

In some embodiments, a preparation method of ergothioneine comprises: mixing the yeast strain described in the first aspect with a fermentation medium and an optional substrate, fermenting under the conditions of the fermentation temperature of 20° C. to 40° C., the airflow rate of 0.8 L/min to 8 L/min, the air pressure of 0 to 0.6 MPa, the pH of 4.0 to 7.0 and the stirring speed of 100 rpm to 900 rpm, supplementing glycerol and increasing the stirring speed stepwise between 100 rpm to 600 rpm on the basis of the initial stirring speed when the dissolved oxygen concentration drops to 50% in the fermentation step, supplementing glycerol continuously and maintaining stepwise increment of the stirring speed until the detected wet weight biomass reaches 200 g/L; then homogenizing cells and separating to obtain ergothioneine; wherein the substrate comprises at least one selected from arginine, histidine, methionine or cysteine; the fermentation medium comprises 1% vol to 7% vol phosphoric acid, 0.03 wt % to 0.3 wt % CaSO₄, 1 wt % to 6 wt % K₂SO₄, 0.1 wt % to 2 wt % KOH, 0.5 wt % to 5 wt % MgSO₄·7H₂O, 1 wt % to 12 wt % glycerol, 0.1 wt % to 2 wt % yeast extract, 0.1 wt % to 2 wt % peptone, 0.01% vol to 1% vol defoaming oil, and the solvent is water.

In some embodiments, a preparation method of ergothioneine comprises: inoculating the yeast strain described in the first aspect into a seed liquid culture medium for amplification culture to obtain a seed liquid, mixing the seed liquid with a fermentation medium and an optional substrate, fermenting under the conditions of the fermentation temperature of 20° C. to 40° C., the airflow rate of 0.8 L/min to 8 L/min, the air pressure of 0 to 0.6 MPa, the pH of 4.0 to 7.0 and the stirring speed of 100 rpm to 900 rpm, supplementing glycerol and increasing the stirring speed stepwise between 100 rpm to 600 rpm on the basis of the initial stirring speed when the dissolved oxygen concentration drops to 50% in the fermentation step, supplementing glycerol continuously and maintaining stepwise increment of the stirring speed until the detected wet weight biomass reaches 200 g/L; then homogenizing cells and separating to obtain ergothioneine; wherein the substrate comprises at least one selected from arginine, histidine, methionine or cysteine; the fermentation medium comprises 1% vol to 7% vol phosphoric acid, 0.03 wt % to 0.3 wt % CaSO₄, 1 wt % to 6 wt % K₂SO₄, 0.1 wt % to 2 wt % KOH, 0.5 wt % to 5 wt % MgSO₄·7H₂O, 1 wt % to 12 wt % glycerol, 0.1 wt % to 2 wt % yeast extract, 0.1 wt % to 2 wt % peptone, 0.01% vol to 1% vol defoaming oil, and the solvent is water.

In some embodiments, a preparation method of ergothioneine comprises: activating the yeast strain described in the first aspect to obtain a culture medium containing the activated yeast strain, inoculating the culture medium containing the activated yeast strain into a seed liquid culture medium for amplification culture to obtain a seed liquid, mixing the seed liquid with a fermentation medium and an optional substrate, fermenting under the conditions of the fermentation temperature of 20° C. to 40° C., the airflow rate of 0.8 L/min to 8 L/min, the air pressure of 0 to 0.6 MPa, the pH of 4.0 to 7.0 and the stirring speed of 100 rpm to 900 rpm, supplementing glycerol and increasing the stirring speed stepwise between 100 rpm to 600 rpm on the basis of the initial stirring speed when the dissolved oxygen concentration drops to 50% in the fermentation step, supplementing glycerol continuously and maintaining stepwise increment of the stirring speed until the detected wet weight biomass reaches 200 g/L; then disrupting cells and separating to obtain ergothioneine.

In the third aspect, the present invention provides an ergothioneine obtained according to the second aspect.

An ergothioneine is obtained according to the preparation method described in the second aspect. The ergothioneine obtained by the preparation method according to the second aspect has fewer impurities, less drug residues, less chemical residues, and high product quality.

In a fourth aspect, the present invention provides a method of preparing ergothioneine comprising using the yeast strain described in the first aspect.

A use of the yeast strain described in the first aspect in preparing ergothioneine.

### Beneficial Effect

Compared with the prior art, an embodiment of the above technical solution has at least one of the following beneficial technical effects:

(1) The yeast strains provided in the present invention can be used to prepare ergothioneine, and the obtained ergothioneine has the advantages of high yield, low cost, fast preparation speed, and excellent industrialization prospects.

(2) The yeast strains provided in the present invention can be passaged many times, and the yield stability of the passaged yeast when preparing ergothioneine is good.

(3) The preparation method of ergothioneine provided in the present invention adopts the method of biological fermentation synthesis. Compared with chemical synthesis and other methods, it does not require expensive raw materials, which is beneficial to greatly reduce the cost and can greatly reduce the use of toxic and harmful chemical reagents in the preparation process. It is environmentally friendly, and is beneficial to reduce the impurities and chemical residues of the product, and is conducive to improving the quality of the product.

(4) Compared with other natural biological extraction methods, the preparation method of ergothioneine provided in the present invention has the advantages of high yield, fewer impurities, less drug residues, and low preparation cost.

(5) Compared with other fermentation technologies, the preparation method of ergothioneine provided in the present invention has the advantages of short fermentation period, high yield, and low cost, which is conducive to industrialized production.

(6) The preparation method provided in the present invention does not need to add organic solvents and toxic and harmful reagents, but is prepared by a biological fermentation method, and the obtained ergothioneine is natural, safe, environmentally friendly, and has no organic solvent residues.

### DESCRIPTIONS OF THE DRAWINGS

FIG. 1 is a schematic diagram of the synthesis of ergothioneine in Example 1.

FIG. 2 is a high performance liquid chromatogram of ergothioneine standard in Example 1.

FIG. 3 is a high performance liquid chromatogram of the fermentation broth of the MM1 strain in Example 1. Compared with FIG. 2, it can be proved that there is ergothioneine in FIG. 3.

FIG. 4 is a schematic diagram of the synthesis of natural ergothioneine in Example 3.

### DEFINITION OF TERMS

In the present invention, regardless of whether the word “approximately”, “about” or “substantially” is used, all numbers disclosed herein are approximate values. Based on the published figures, the value of each number may have a difference of ±10% or less or a reasonable difference considered by those in the art, such as a difference of ±1%, ±2%, ±3%, ±4%, or ±5%.

The term “and/or” should be understood to mean any one of the alternatives or a combination of any two or more of the alternatives.

The term “option”, “optional” or “optionally” means that the subsequently described event or situation can but does not necessarily occur. For example, “mixing the yeast strain described in the first aspect with a fermentation medium and an optional substrate” means “mixing the yeast strain described in the first aspect with a fermentation medium and a substrate” or “mixing the yeast strain described in the first aspect with a fermentation medium”.

The term “inoculation amount” refers to the volume percentage of the volume of the culture medium containing the activated yeast strain to the total volume of the seed liquid culture medium and the culture medium of the activated yeast strain after the inoculation; or the volume percentage of the volume of the seed liquid to the total volume of the seed liquid and the fermentation medium after the seed liquid is inoculated into the fermentation medium.

The term “OD₆₀₀” refers to the detected optical density at a wavelength of 600 nm.

The term “wt %” refers to mass percentage.

The term “% vol” refers to volume percentage.

The term “SAM” refers to S-adenosylmethionine, which is a naturally occurring intermediate metabolite in cells.

The term “SAH” refers to S-adenosylhomocysteine, which is produced by demethylation of SAM and is a naturally occurring intermediate metabolite in cells.

Reference throughout this specification to “an embodiment”, “some embodiments”, “one embodiment”, “another example”, “an example”, “a specific example” or “some examples” means that a particular feature, structure, material, or characteristic described in connection with the embodiment or example is included in at least one embodiment or example of the present disclosure. Thus, the appearances of the phrases such as “in some embodiments”, “in one embodiment”, “in an embodiment”, “in another example”, “in an example”, “in a specific example” or “in some examples” in various places throughout this specification are not necessarily referring to the same embodiment or example of the present disclosure. Furthermore, the particular features, structures, materials, or characteristics may be combined in any suitable manner in one or more embodiments or examples. In addition, those skilled in the art can integrate and combine different embodiments, examples or the features of them as long as they are not contradictory to one another.

### EXAMPLES

In order to enable those skilled in the art to better understand the technical solutions herein, some non-limiting embodiments are further disclosed below to further describe the present invention in detail.

The reagents used herein can be purchased from the market or can be prepared by the method described in this invention.

Unless otherwise specified, the formula of each medium described in the following specific examples is as follows:

The formula of the YPD medium (yeast extract peptone dextrose medium) is: yeast extract 10 g/L, peptone 10 g/L and dextrose 20 g/L, and the solvent is water.

The formula of the YPD agar medium (yeast extract peptone dextrose agar medium) is: yeast extract 10 g/L, peptone 10 g/L, dextrose 20 g/L and agar powder 15 g/L, and the solvent is water.

The formula of the YPD agar medium containing 0.02% lithium chloride (as a mutagen) is: yeast extract 10 g/L, peptone 10 g/L, dextrose 20 g/L, agar powder 15 g/L and lithium chloride 0.2 g/L, and the solvent is water.

The combined treatments of physical and chemical mutagenic agents breeding technology described in this application is a conventional mutagenesis method well known to those skilled in the art. It should be understood that the method is only used to illustrate the application, and cannot limit the scope of protection of the application. For those skilled in the art, without departing from the spirit and essence of the application, various changes or modifications to the medium components, content, culture conditions, and mutation conditions in the method also fall within the protection scope of the application.

Preferably, in the ultraviolet (UV) mutation described in this application, the yeast strain suspension is irradiated at 25-35 cm under an ultraviolet lamp for 15-25 minutes. Preferably, in the lithium chloride mutation described in this application, a yeast strain suspension is spread to a medium containing 0.01-0.02% lithium chloride (as a mutagen), and cultured at 18-37° C. for 16-72 hours.

Specifically, the yeast strain suspension is prepared by: taking Saccharomyces cerevisiae, Pichia pastoris, Schizosaccharomyces pombe, Torulaspora delbrueckii or Kluyveromyces lactis broth cultured at 18-37° C. for 16-72 hours, the cells in the broth are collected by centrifugation and washed 2-3 times with sterile water, then an appropriate amount of Tween 80 is added, the mixture is resuspended and shaken to disperse the cells, the cell density is adjusted to about 1,000,000 cells/mL with sterile water.

The term “rpm” means the speed “revolutions per minute”; “° C.” means the temperature unit “degrees Celsius”; “L” means the volume unit “liter”; “mL” means the volume unit “milliliters”; “pH” means pH degree of acid or alkali; “g” means the mass unit “gram”; “Mpa” means the pressure unit “megapa”.

### Example 1

### Screening of Yeast Strains

Preparation of Cell Suspension

The isolated Saccharomyces cerevisiae, Pichia pastoris, Schizosaccharomyces pombe, Torulaspora delbrueckii or Kluyveromyces lactis was inoculated into a 50 mL flat-bottomed Erlenmeyer flask containing 10 mL of yeast extract peptone dextrose (YPD) medium, and grown at 28° C. and 250 rpm for 48 hours. The yeast cells in the broth were collected by centrifugation and washed 3 times with 10 mL of sterile water, then resuspended with 10 mL of sterile water and placed in a 50 mL flat-bottomed Erlenmeyer flask. 5 μL of Tween 80 was added with a pipette, and the resulting mixture was shaken at 28° C. and 250 rpm for 30 min to disperse the cells. The cell density of each yeast strain was obtained by detecting OD₆₀₀, and an appropriate amount of sterile water was added to adjust the cell density to about 1,000,000 cells/mL. UV mutation

10 mL of each yeast strain suspension with a cell density of about 1,000,000 cellsmL was taken and placed in a sterilized petri dish with a diameter of 90 mm. The petri dishes were placed on the stage of the magnetic stirrer, the lids of the dishes were opened, and the ultraviolet irradiation was performed 30 cm below the ultraviolet lamp, and the irradiation time was 20 min.

Lithium Chloride Mutation

10 mL of each yeast strain suspension after UV mutation was taken and all were spread on YPD agar medium containing 0.02% lithium chloride. They were wrapped with kraft paper, and placed in a 28° C. incubator for 72 hours.

Screening of Yeast Strains

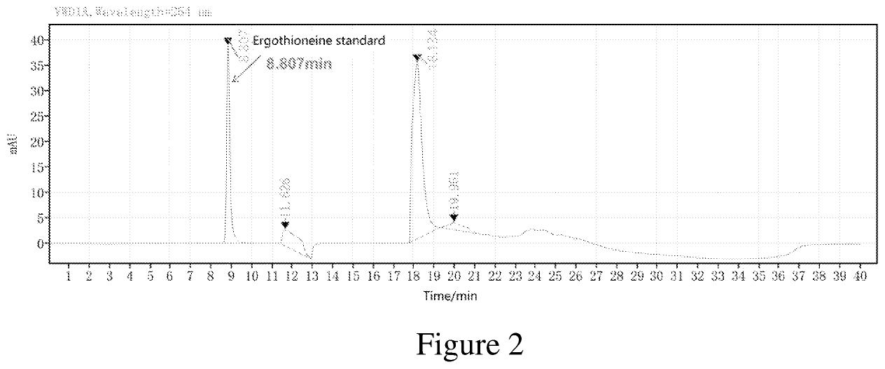

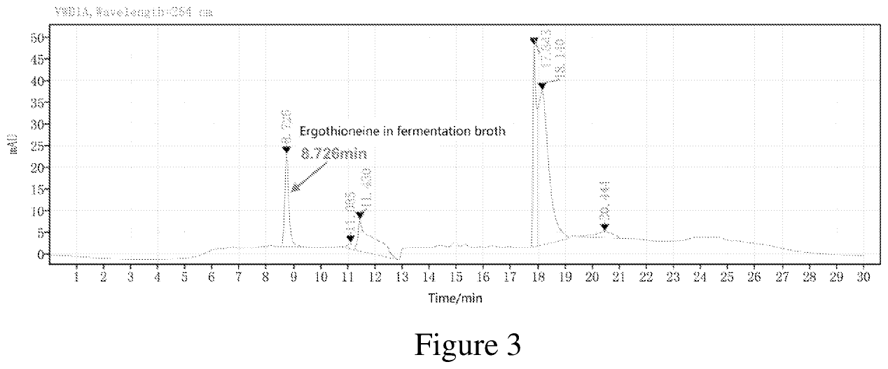

Well-growing mutant strains of each yeast strain with relatively large single colony diameters that had undergone UV mutation and lithium chloride mutation were selected and streaked on the YPD agar medium plates. They were cultured in a constant temperature incubator at 28° C. for 48 hours. About 500 mutant strains were picked for each yeast strain. Each mutant strain was inoculated into a 50 mL flat-bottomed Erlenmeyer flask containing 10 mL of YPD medium, and grown at 28° C. and 250 rpm for 48 hours to obtain a broth. The ergothioneine-containing supernatant was collected by homogenizing the cells and centrifugation, the ergothioneine standard and the supernatant were taken, and the content of ergothioneine obtained by fermentation in the supernatant was determined by high performance liquid chromatography (HPLC), as shown in FIG. 2 and FIG. 3 respectively. The solution of each yeast strain before the combined treatments of physical and chemical mutagenic agents was taken as a control.

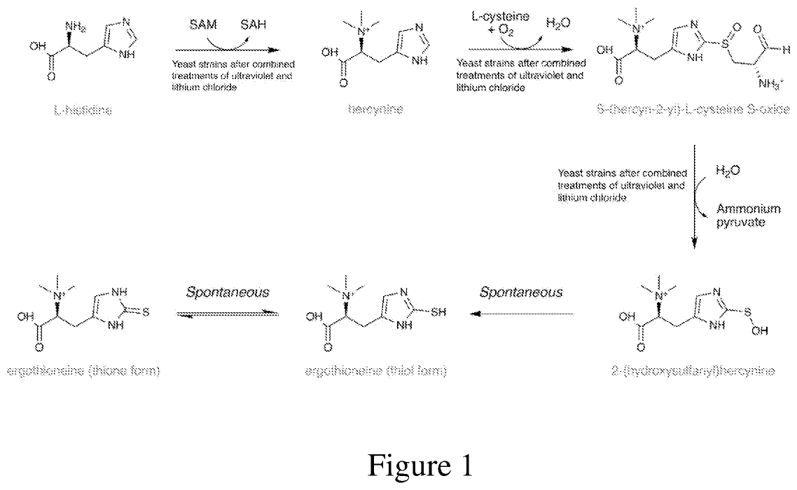

Before the combined treatments of ultraviolet and lithium chloride, no ergothioneine was detected in each yeast strain by HPLC. After the combined treatments of ultraviolet and lithium chloride, some yeast strains were detected to have the ability to synthesize ergothioneine, and a Schizosaccharomyces pombe strain that could accumulate a certain amount of ergothioneine in cells was selected and named MM1. The yield of ergothioneine (that is, the content of ergothioneine in the supernatant) was about 62.6 mg/L, as shown in FIG. 1. The mutated Schizosaccharomyces pombe strain MM1 was stored at minus 80° C. with glycerol and was passaged 5 times, and the yield of ergothioneine was stable.

### Example 2

### Multiple Round of Screening of Schizosaccharomyces pombe Strain MM1 Preparation of Cell Suspension

The Schizosaccharomyces pombe strain MM1 was inoculated into a 50 mL flat-bottomed Erlenmeyer flask containing 10 mL of yeast extract peptone dextrose (YPD) medium separately, and grown at 28° C. and 250 rpm for 48 hours. The yeast cells in the broth were collected by centrifugation and washed 3 times with 10 mL of sterile water, then resuspended with 10 mL of sterile water and placed in a 50 mL flat-bottomed Erlenmeyer flask. 5 μL of Tween 80 was added with a pipette, and the resulting mixture was shaken at 28° C. and 250 rpm for 30 min to disperse the cells. The cell density of Schizosaccharomyces pombe strain MM1 was obtained by detecting OD₆₀₀, and an appropriate amount of sterile water was added to adjust the cell density to about 1,000,000 cells/mL.

UV Mutation

10 mL of Schizosaccharomyces pombe strain MM1 suspension with a cell density of about 1,000,000 cellsmL was taken separately and placed in a sterilized petri dish with a diameter of 90 mm. The petri dish was placed on the stage of the magnetic stirrer, the lid of the dish was opened, and the ultraviolet irradiation was performed 30 cm below the ultraviolet lamp, and the irradiation time was 20 min.

Lithium Chloride Mutation

10 mL of Schizosaccharomyces pombe strain MM1 suspension after UV mutation was taken separately and all was spread on YPD agar medium containing 0.02% lithium chloride (as a mutagen). It was wrapped with kraft paper, and placed in a 28° C. incubator for 72 hours.

Screening of Schizosaccharomyces pombe

500 well-growing mutant strains of Schizosaccharomyces pombe strain MM1 with relatively large single colony diameters that had undergone UV mutation and lithium chloride mutation were selected and streaked on the YPD agar medium plates. They were cultured in a constant temperature incubator at 28° C. for 48 hours. The mutant strains were inoculated into a 50 mL flat-bottomed Erlenmeyer flask containing 10 mL of YPD medium, and grown at 28° C. and 250 rpm for 48 hours to obtain a broth. The ergothioneine-containing supernatant was collected by homogenizing the cells and centrifugation, the ergothioneine standard and the supernatant were taken, and the content of ergothioneine in the supernatant was determined by high performance liquid chromatography (HPLC). A solution containing Schizosaccharomyces pombe strain MM1 before multiple round of screening was used as a control.

After the combined treatments of ultraviolet and lithium chloride, a Schizosaccharomyces pombe strain that could accumulate a certain amount of ergothioneine in cells was selected and named MM2. The yield of ergothioneine (that is, the content of ergothioneine in the supernatant) was about 598 mg/L. The mutated Schizosaccharomyces pombe strain MM2 was stored at minus 80° C. with glycerol and was passaged 5 times, and the yield of ergothioneine was stable.

The Schizosaccharomyces pombe strain MM2 was subjected to the combined treatments of ultraviolet and lithium chloride again. After multiple round of the combined treatments, a Schizosaccharomyces pombe strain with high yield of ergothioneine was finally selected and named OMK-79 (The deposit number of this strain is CCTCC M 20211505, and it was deposited biologically at China Center for Type Culture Collection (CCTCC, Wuhan University) on Nov. 29, 2021). The mutated Schizosaccharomyces pombe strain OMK-79 was stored at minus 80° C. with glycerol and was passaged 5 times, and the yield of ergothioneine was stable.

### Example 3

### Fermentation Production of Ergothioneine

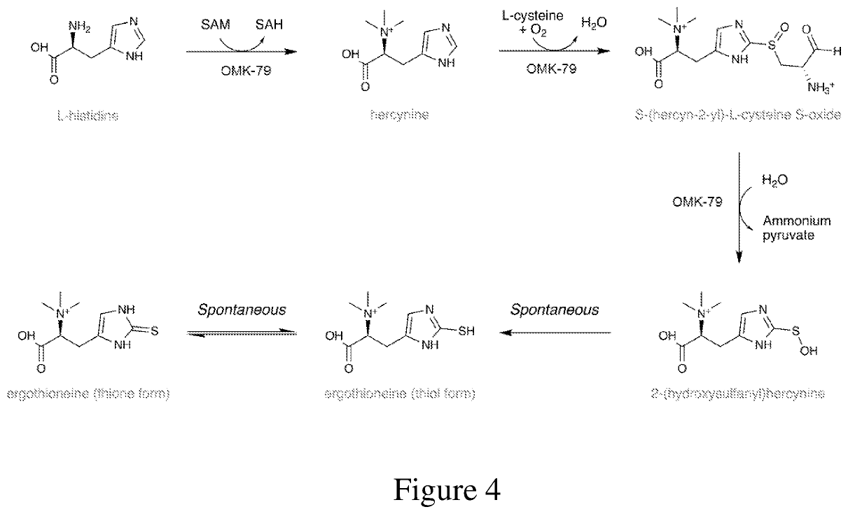

The synthesis process shown in FIG. 4 specifically includes the following steps:

The Schizosaccharomyces pombe strain OMK-79 was inoculated into a 250 mL flat-bottomed Erlenmeyer flask containing 50 mL of YPD medium, and grown at 28° C. and 250 rpm for 48 hours to activate the strain. The activated OMK-79 strain was transferred to a 1 L flat-bottomed Erlenmeyer flask containing 150 mL of YPD medium at an inoculation amount of 1%, and grown at 28° C. and 250 rpm for 18 hours to obtain a seed liquid. 100 mL of Schizosaccharomyces pombe OMK-79 seed liquid was inoculated into 3 L of fermentation medium for fermentation. The fermentation was carried out in a 5 L small-scale bioreactor, and its temperature and pH were controlled at 28-30° C. and 4.0-7.0, respectively. During the whole operation period of the above fermentation, the airflow rate was maintained at 3 L/min, the air pressure was maintained at 0.08 Mpa, the pH was controlled by adding ammonia water, and the initial stirring speed was 200 rpm.

When the dissolved oxygen concentration (DO) dropped to 50% (about 24 hours after fermentation), glycerol began to be supplemented and the stirring speed was increased stepwise to 600 rpm until the detected biomass reached 200 g/L (wet weight), the total supplemented amount of glycerol was about 75 g/L. The cell density of the fermentation broth reached saturation 120 hours after supplementing glycerol, and the OD₆₀₀ value could reach up to 400. During the entire fermentation period after the start of the supplementation of glycerol, the airflow rate was maintained at 4 L/min, and the air pressure was maintained at 0.10 Mpa. After the fermentation, the ergothioneine-containing supernatant was collected by homogenizing the cells and centrifugation. The content of ergothioneine in the supernatant was determined by high performance liquid chromatography (HPLC), and the content was 7.6 g/L.

### Example 4

### Production Optimization of Ergothioneine

The Schizosaccharomyces pombe strain OMK-79 was inoculated into a 250 mL flat-bottomed Erlenmeyer flask containing 50 mL of YPD medium, and grown at 28° C. and 250 rpm for 48 hours to activate the strain. The activated OMK-79 strain was transferred to a 1 L flat-bottomed Erlenmeyer flask containing 150 mL of YPD medium at an inoculation amount of %, and grown at 28° C. and 250 rpm for 18 hours to obtain a seed liquid. 100 mL of Schizosaccharomyces pombe OMK-79 seed liquid was inoculated into 3 L of fermentation medium for fermentation. The fermentation was carried out in a 5 L small-scale bioreactor, and its temperature and pH were controlled at 28-30° C. and 5.0-6.0, respectively. During the whole operation period of the above fermentation, the airflow rate was maintained at 3 L/min, the air pressure was maintained at 0.08 Mpa, the pH was controlled by adding ammonia water, and the initial stirring speed was 200 rpm.

When the dissolved oxygen concentration (DO) dropped to 50% (about 24 hours after fermentation), glycerol and substrate began to be supplemented. The substrate was 1 g/L arginine, 1 g/L histidine, 1 g/L methionine or 1 g/L cysteine. After starting to supplement glycerol and substrate, the stirring speed was gradually increased to 600 rpm until the detected biomass reached 200 mg/mL (wet weight). The supplemented amount of glycerol was about 75 g/L. The cell density of the fermentation broth reached saturation 120 hours after supplementing glycerol and substrate, and the OD₆₀₀ value could reach up to 400. During the entire fermentation period after the start of the supplementation of glycerol and substrate, the airflow rate was maintained at 4 L/min, and the air pressure was maintained at 0.10 Mpa. After the fermentation, the ergothioneine-containing supernatant was collected by homogenizing the cells and centrifugation. The content of ergothioneine in the supernatant was determined by high performance liquid chromatography (HPLC). Through experiments, the supplementation of the substrates arginine, histidine, methionine and cysteine all promoted the production of ergothioneine, and the content of ergothioneine in the supernatant was 10.7 g/L (substrate was arginine), 9.8 g/L (substrate was histidine), 9.2 g/L (substrate was methionine) and 8.4 g/L (substrate was cysteine).

### Example 5

### Fermentation Optimization of Ergothioneine

The Schizosaccharomyces pombe strain OMK-79 was inoculated into a 250 mL flat-bottomed Erlenmeyer flask containing 50 mL of YPD medium, and grown at 28° C. and 250 rpm for 48 hours to activate the strain. The activated OMK-79 strain was transferred to a 1 L flat-bottomed Erlenmeyer flask containing 150 mL of YPD medium at an inoculation amount of 1%, and grown at 28° C. and 250 rpm for 18 hours to obtain a seed liquid. 100 mL of Schizosaccharomyces pombe OMK-79 seed liquid was inoculated into 3 L of fermentation medium for fermentation. The fermentation was carried out in a 5 L small-scale bioreactor, and its temperature and pH were controlled at 28-30° C. and 5.0-6.0, respectively. During the whole operation period of the above fermentation, the airflow rate was maintained at 3 L/min, the air pressure was maintained at 0.08 Mpa, the pH was controlled by adding ammonia water, and the initial stirring speed was 200 rpm.

When the dissolved oxygen concentration (DO) dropped to 50% (about 24 hours after fermentation), glycerol, 1.3 g/L arginine, 0.8 g/L histidine, 0.6 g/L methionine and 0.5 g/L cysteine began to be supplemented. After starting to supplement glycerol and substrate, the stirring speed was gradually increased to 600 rpm until the detected biomass reached 200 mg/mL (wet weight). The supplemental amount of glycerol was about 75 g/L. The cell density of the fermentation broth reached saturation 120 hours after supplementing glycerol and substrate, and the OD₆₀₀ value could reach up to 400. During the entire fermentation period after the start of the supplementation of glycerol and substrate, the airflow rate was maintained at 4 L/min, and the pressure was maintained at 0.10 Mpa. After about 148 hours of fermentation, the ergothioneine-containing supernatant was collected by homogenizing the cells and centrifugation. The content of ergothioneine in the supernatant was determined by high performance liquid chromatography (HPLC), and the content was 12.5 g/L.

The method of this application has been described through the preferred embodiments. It is obvious that relevant persons can make changes or appropriate changes and combinations to the methods and applications described herein within the content, spirit and scope of this application to realize and apply the technology of this application. Those skilled in the art can learn from the content of this article and appropriately improve the process parameters to achieve. In particular, it should be pointed out that all similar replacements and modifications are obvious to those skilled in the art, and they are all deemed to be included in this application.

What is claimed is:

1. A yeast strain with the deposit number of CCTCC M 20211505.

2. A preparation method of ergothioneine, comprising: mixing the yeast strain according to claim 1 with a fermentation medium and an optional substrate, fermenting, and then homogenizing cells and separating to obtain ergothioneine.

3. The preparation method according to claim 2, wherein the yeast strain is first inoculated into a seed liquid culture medium for amplification culture to obtain a seed liquid, then the seed liquid is mixed with a fermentation medium and an optional substrate, fermenting, and then the cells are homogenized and separated to obtain ergothioneine.

4. The preparation method according to claim 2, wherein the yeast strain is first activated to obtain a culture medium containing the activated yeast strain, then the culture medium containing the activated yeast strain is inoculated into a seed liquid culture medium for amplification culture to obtain a seed liquid, and the seed liquid is mixed with a fermentation medium and an optional substrate, fermenting, and then the cells are homogenized and separated to obtain ergothioneine.

5. The preparation method according to claim 2, wherein the substrate comprises at least one selected from arginine, histidine, methionine or cysteine.

6. The preparation method according to claim 2, wherein the fermentation medium comprises a carbon source.

7. The preparation method according to claim 6, wherein the carbon source includes at least one selected from sucrose, fructose, xylose, ethanol, methanol, glycerol, glucose, cellulose, starch, cellobiose or other glucose-containing polymers.

8. The preparation method according to claim 4, wherein the fermentation medium comprises phosphoric acid, CaSO₄, K₂SO₄, KOH, MgSO₄·7H₂O, glycerol, yeast extract, peptone, defoaming oil and water.

9. The preparation method according to claim 8, wherein the fermentation medium comprises 1% vol to 7% vol phosphoric acid, 0.03 wt % to 0.3 wt % CaSO₄, 1 wt % to 6 wt % K₂SO₄, 0.1 wt % to 2 wt % KOH, 0.5 wt % to 5 wt % MgSO₄·7H₂O, 1 wt % to 12 wt % glycerol, 0.1 wt % to 2 wt % yeast extract, 0.1 wt % to 2 wt % peptone, 0.01% vol to 1% vol defoaming oil, and the solvent is water.

10. The preparation method according to claim 9, wherein the fermentation medium comprises 2.27% vol phosphoric acid, 0.093 wt % CaSO₄, 1.82 wt % K₂SO₄, 0.413 wt % KOH, 1.49 wt % MgSO₄·7H₂O, 4 wt % glycerol, 0.5 wt % yeast extract, 0.5 wt % peptone, 0.1% vol defoaming oil, and the solvent is water.

11. The preparation method according to claim 4, wherein the seed liquid culture medium comprises yeast extract, peptone and glucose.

12. The preparation method according to claim 4, wherein the seed liquid culture medium comprises yeast extract 3 g/L to 30 g/L, peptone 3 g/L to 30 g/L, and glucose 5 g/L to 50 g/L.

13. The preparation method according to claim 4, wherein the seed liquid culture medium comprises yeast extract 10 g/L, peptone 10 g/L, and glucose 20 g/L.

14. The preparation method according to claim 2, wherein the fermentation temperature is 20° C. to 40° C.; and/or
the fermentation time is 16 hours to 200 hours; and/or
the fermentation has an airflow rate of 0.8 L/min-8 L/min; and/or
the air pressure of the fermentation is 0 to 0.6 MPa; and/or
the pH of the fermentation system of the fermentation is 4.0 to 7.0; and/or
the initial stirring speed of the fermentation is between 100 rpm to 900 rpm.

15. The preparation method according to claim 2, when the dissolved oxygen concentration drops to 50% during the fermentation, glycerol is supplemented, and on the basis of the initial stirring speed, the stirring speed is increased stepwise between 100 rpm to 600 rpm; glycerol is supplemented continuously and the stirring speed maintains stepwise increment until the detected wet weight biomass reaches 30 g/L to 300 g/L.

16. The preparation method according to claim 2, wherein the supplemented amount of glycerol is 20 g/L to 220 g/L.

17. The preparation method according to claim 2, comprising: inoculating the yeast strain into a seed liquid culture medium for amplification culture to obtain a seed liquid, mixing the seed liquid with a fermentation medium and an optional substrate, fermenting under the conditions of the fermentation temperature of 20° C. to 40° C., the airflow rate of 0.8 L/min to 8 L/min, the air pressure of 0 to 0.6 MPa, the pH of 4.0 to 7.0 and the stirring speed of 100 rpm to 900 rpm; supplementing glycerol and increasing the stirring speed stepwise between 100 rpm to 600 rpm on the basis of the initial stirring speed when the dissolved oxygen concentration drops to 50% during the fermentation; supplementing glycerol continuously and maintaining stepwise increment of the stirring speed until the detected wet weight biomass reaches 200 g/L; then homogenizing cells and separating to obtain ergothioneine; wherein the substrate comprises at least one selected from arginine, histidine, methionine or cysteine; the fermentation medium comprises 1% vol to 7% vol phosphoric acid, 0.03 wt % to 0.3 wt % CaSO₄, 1 wt % to 6 wt % K₂SO₄, 0.1 wt % to 2 wt % KOH, 0.5 wt % to 5 wt % MgSO₄·7H₂O, 1 wt % to 12 wt % glycerol, 0.1 wt % to 2 wt % yeast extract, 0.1 wt % to 2 wt % peptone, 0.01% vol to 1% vol defoaming oil, and the solvent is water.

18. The preparation method according to claim 17, wherein the yeast is first inoculated in the seed culture medium, grown at 28° C. and 250 rpm for 48 hours for activation, then the culture medium containing the activated yeast strain is inoculated in the seed solution culture medium for expanded culture to obtain the seed solution.

19. The preparation method according to claim 18, wherein the seed liquid culture medium comprises yeast extract 3 g/L to 30 g/L, peptone 3 g/L to 30 g/L, and glucose 5 g/L to 50 g/L.
